(19) United States
(12) Patent Application Publication
(10) Pub. No.: US 2008/0289640 A1
(43) Pub. Date: Nov. 27, 2008

(54) VACUUM ACTIVATED DEVICE FOR HUMAN IMMOBILIZATION

(76) Inventors: Chris Kline, Austin, TX (US); Nathan Anderson, Austin, TX (US); Nathan Nuttall, Austin, TX (US)

Correspondence Address: Chris Kline, 4910 Pelham, Austin, TX, 78727, US

(21) Appl. No.: 12/124,069

(22) Filed: May 20, 2008

Related U.S. Application Data
(60) Provisional application No. 60/939,114, filed on May 21, 2007.

(51) Int. Cl. A61B 19/00

(52) U.S. Cl. 128/869

(57) ABSTRACT

A vacuum activated device for holding a human immobile. The device includes a rigid frame of a three dimensional shape large enough to surround a human torso and a bag comprised of a flexible, air impermeable material enclosing the frame. A closure on the bag is necessary to allow an occupant to enter or be placed within it. When air is evacuated through a hole in the bag its surface will collapse around the frame and the occupant's body. The rigidity of the frame in combination with the pressure applied to the occupant by the surface of the bag will impede further movement by the occupant. A means to allow the occupant access to breathable air will be provided.

### CROSS-REFERENCE TO RELATED APPLICATIONS

This invention claims the benefit of PPA Ser. No. 60/939,114, filed 2007 May 21 by the present inventors, which is incorporated by reference.

### BACKGROUND

1. Field of Invention

This invention relates to mechanisms and methods for holding a person immobile. Restraints of this nature are usually categorized by their uses in a medical, sexual, criminal, or military context.

2. Prior Art

A person can be held significantly immobile with relatively simple restraints but often they allow for undesirable consequences. Common restraints such as ropes or cuffs bind specific areas on the human body such as wrists or ankles. These methods typically rely on the limited range of motion of the limbs and torso to achieve immobility. The restrained is able to struggle against the bound areas which may result in injury or escape.

Some restraints have overcome the risk of the restrained focusing their efforts by affecting the entire body. An example of such a human immobilization invention which can restrain without binding specific areas is U.S. Pat. No. 4,202,279 for Sticky Foam. This foam unfortunately creates new consequences including a substantial removal process and the risk of obstructing breathing passages. In the medical field some inventions for immobilizing patients have used beads within an air impermeable bag which lock in place evenly around the restrained when air is evacuated, such as U.S. Pat. No. 3,212,497 filed 1963 and U.S. Pat. No. 3,762,404 filed 1971.

Devices for deforming a flexible skin around an object utilizing air pressure have been in use for decades in the context of packaging. These are numerous enough to have their own category in the USPTO, a quintessential example being U.S. Pat. No. 2,289,668 filed in 1940. However, these packaging devices typically require machinery, temperatures, or chemicals which are incompatible with the human body. Further, without a rigid element to complement a vacuumed skin it will allow undesired mobility when applied to restraining a person.

### SUMMARY

In most embodiments there will be a hollowed frame forming a simple three-dimensional structure large enough to enclose an occupant. It will be covered with a bag made of a flexible, air-impermeable material. The bag and frame will have an opening into which a vacuum can be applied. An occupant will enter or be placed within the bag. When a vacuum is applied it will cause the bag to deform around both the rigid frame and the occupant thus rendering the occupant immobile. The bag can have more openings to allow access to the immobilized individual inside or to allow portions of the occupant to remain outside of the device. The occupant will obtain breathable air by means such as a breathing tube, aqualung, or placement of their head through an opening in the bag.

### DRAWINGS

### Figures

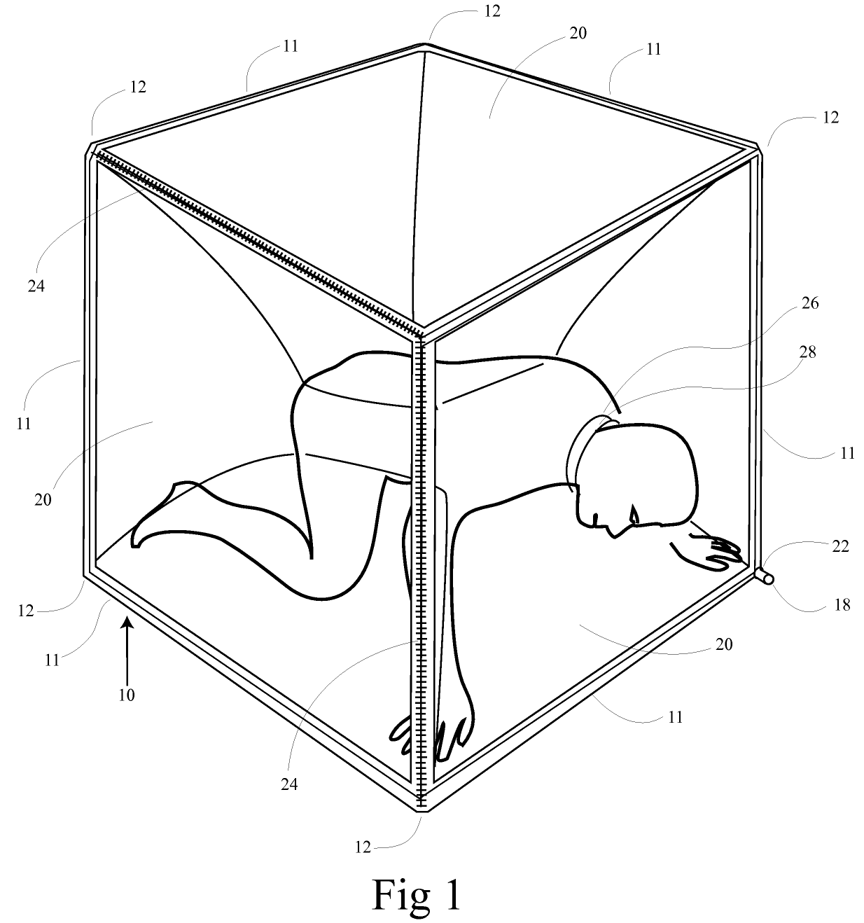

FIG. 1 is a perspective view of the device in operation with an immobilized occupant.

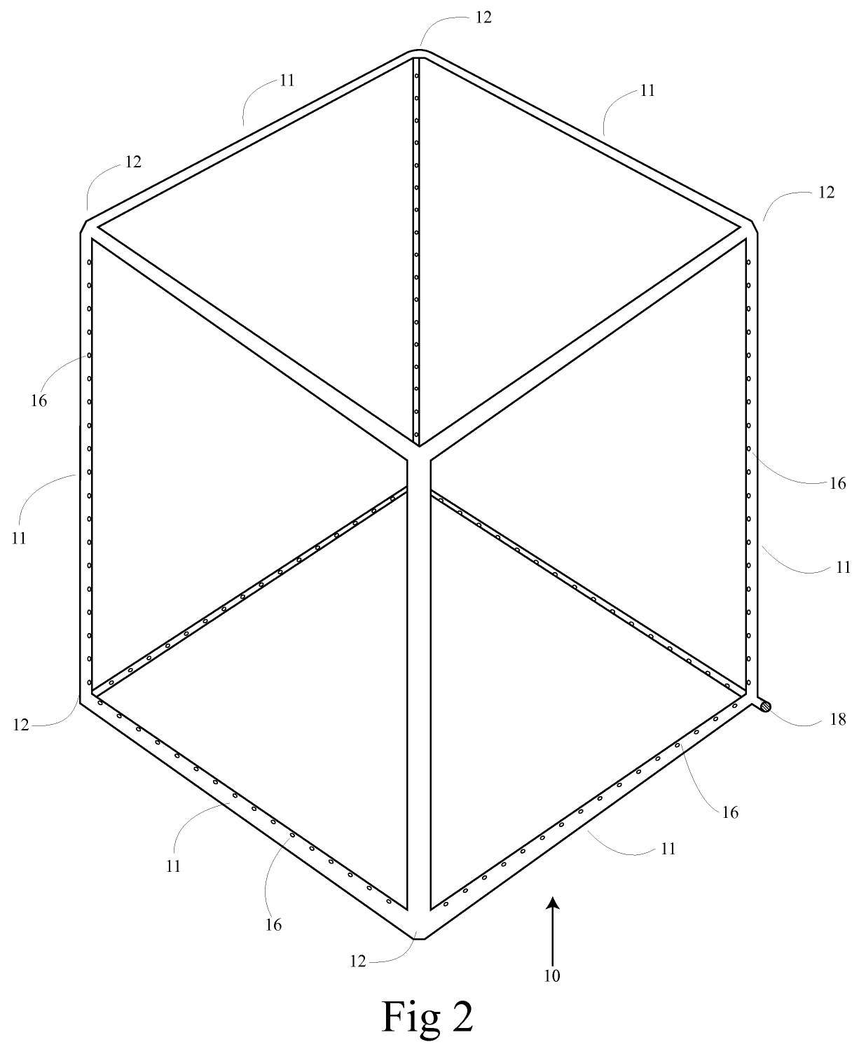

FIG. 2 is a perspective view of the frame only, without the enclosing bag.

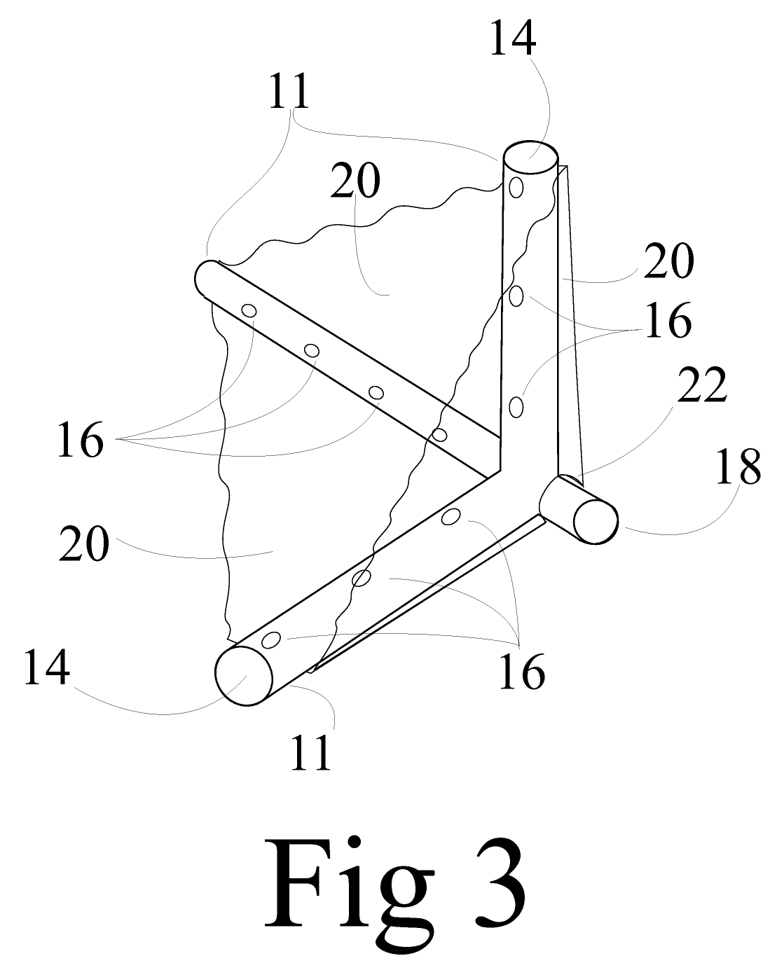

FIG. 3 is a partial cut-away view showing the corner of the frame where a vacuum would be applied.

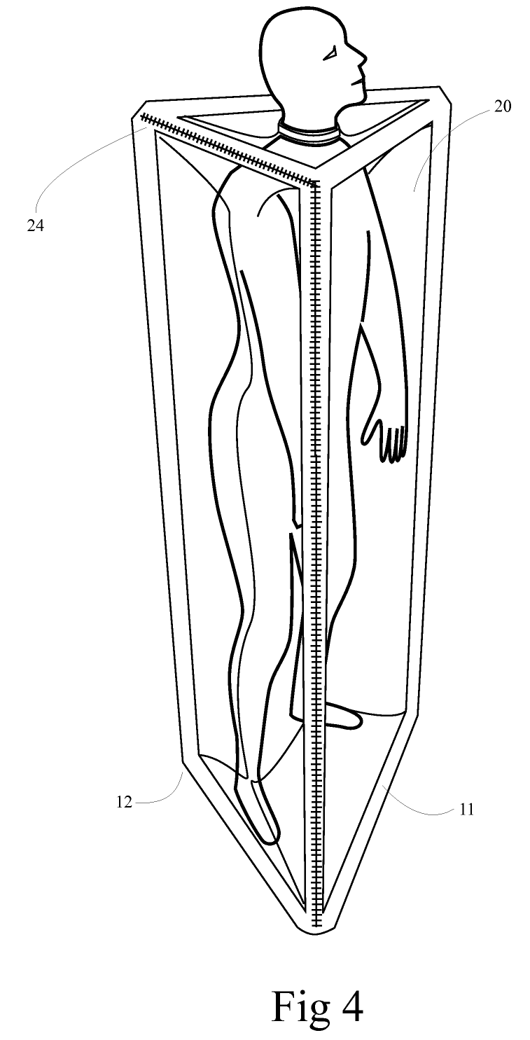

FIG. 4 is a perspective view of a prism-shaped embodiment in operation with an immobilized occupant.

### DRAWINGS—REFERENCE NUMERALS

10 frame
11 rigid elongated support members of frame
12 corner joints of frame
14 hollow center of support members
16 openings in support members
18 protrusion from frame
20 bag whick encloses frame
22 hole in bag for protrusion
24 closure in bag for occupant's neck
26 tube for occupant's neck

### DETAILED DESCRIPTION

### First Embodiment—FIGS. 1-3

FIG. 1 shows an embodiment in which the frame 10 forms the shape of a cube. The size can vary widely provided it is large enough to enclose an average human torso. For this embodiment the cube will measure roughly 1.5 meters in each dimension. FIG. 1 shows this embodiment in operation holding a person immobile in a crouched position. The frame 10 is comprised of elongated support members 11 and joints 12 which can be comprised of any relatively rigid material such as plastic, metal, or wood. ANSI Schedule 40 PVC piping is a convenient material for the frame 10 because it allows for the support members 11 and joints 12 to simply be pushed together for ease of assembly and disassembly. The rigid elongated support members 11 in this embodiment are 12 sections of PVC pipe cut to the same length which define the edges of the frame 10. PVC corners 12 which join the edges are commonly available as plumbing or furniture supplies. FIG. 2 displays the frame 10 on its own without a bag or occupant. Permanently joining the frame 10 parts is not necessary since the force exerted on the frame 10 during operation compresses the edges and joints together. The frame 10 is hollow 14 and the support members 11 have many openings 16 to allow for air to flow through from the inside of the cube to the source of the vacuum.

One corner of the frame 10 has an extra protrusion 18 where the vacuum should be applied. FIG. 3 shows a cut-away view of said corner. In the preferred embodiment the outside diameter of the protrusion 18 is approximately 1.9 cm to interface many commercially available vacuum cleaners. A necessary amount of vacuum for immobilization can be produced by a range of common, commercially available vacuum cleaners.

The frame 10 is covered by a bag 20 of roughly the same shape and surface area as the faces of the cube. The bag 20 is unattached to the frame 10 until assembly. The bag 20 should be made from an air-impermeable flexible material. Latex rubber sheeting of 0.5 mm thickness is an excellent material for the bag 20 because of its high elasticity and tensile strength.

Some manner of closure 24 such as a zipper is necessary along one or more edges of the bag to provide access to the inside. An extra flap of air impermeable material may be used to reduce airflow through the closure 24 when it is closed, however an air-tight seal is not necessary if a vacuum can be continuously applied.

A hole 22 in the corner of the bag 20 allows for the air inside the bag 20 to interface a vacuum. The hole 22 in the bag is matched in diameter and position to the hollow protrusion from the frame 18.

A larger hole 26 in one face of the bag 20 will allow for the occupant's head to remain outside the bag 20 to prevent suffocation. A tube 28 of the same material as the bag 20 approximately 12 cm in diameter and 7-15 cm in length should be attached to the larger hole 26 to better interface with the occupant's neck and prevent air from leaking through this aperture.

### Operation—FIG. 1

If less than 3 edges have a closure 24 the frame should be assembled inside the bag 20. The openings 16 on the support members 11 of the frame 10 should be facing towards the center of the frame 10 to facilitate the vacuum. Once the frame 10 is assembled the bag 20 should approximately match its shape. The smaller hole 22 should closely interface the protrusion 18 to facilitate a significantly air-tight seal between the vacuum source and the inside of the bag 20. The occupant will enter the bag 20 after opening the closure 24. After the occupant enters the bag 20 it will be re-closed from the outside. The occupant should then place their head and neck through the larger hole 26 and tube 28. Once the occupant's head is safely outside the device a vacuum can be applied at the protrusion 18. Many commercially available vacuum cleaners can be used to produce a sufficient vacuum. When a vacuum is applied the bag 20 will deflate and the faces of the bag 20 will conform to the shape of the occupant, causing immobility. The edges of the bag 20 will remain in place around the frame 10, thus providing structure and stability. If the protrusion 18 is sealed at this time and the closure 24 is air-tight the vacuum need not be continuously applied and the device can be freely moved.

### Description and Operation of Alternative Embodiments—FIG. 4

Alternative embodiments could utilize frames of various sizes or shapes. For example, a prism, cylinder, or sphere tall enough for the occupant to stand upright within. Otherwise the description and operation would be the same for these embodiments. Such an alternative embodiment is pictured in FIG. 4. Alternative methods of allowing the occupant access to breathable air such as an aqualung or a breathing tube would allow for the occupant's head to be within the bag. This would eliminate the need for the large hole 26 and tube 28 for the occupant's head.

1. A vacuum activated device for human restraint comprising:
(a) a substantially rigid frame of a three dimensional shape large enough to enclose a human torso,
(b) a bag made of a substantially flexible, air impermeable material formed approximately into said three dimensional shape which encloses said frame,
(c) a means for an occupant to enter or be placed within said bag,
(d) a means to allow the occupant access to breathable air, and
(e) a means for evacuating air from within said bag.

2. The system of claim 1 wherein said frame comprises:
(a) a plurality of hollow elongated support members containing a multitude of through-holes in the outer wall,
(b) a means for joining said elongated support members thereby defining an air passage within said frame,
(c) a hollow protrusion from the frame which interfaces a hole in said bag and communicates with said air passage and said means for evacuating air from said bag.
